(19) United States
(12) Patent Application Publication
(10) Pub. No.: US 2023/0317268 A1
(43) Pub. Date: Oct. 5, 2023

(54) DEVICE, SYSTEM AND METHOD FOR ASSISTING IN THE TREATMENT OF A PATIENT

(71) Applicant: CLINOMIC GMBH, Aachen (DE)

(72) Inventors: Arne PEINE, Aachen (DE); Lukas MARTIN, Aachen (DE)

(21) Appl. No.: 18/005,687

(22) PCT Filed: Jul. 7, 2021
(86) PCT No.: PCT/EP2021/068900

§ 371 Date: Jan. 17, 2023

(30) Foreign Application Priority Data
Jul. 17, 2020 (DE) 10 2020 118 966.4
Jul. 23, 2020 (EP) 20187293.4
Feb. 11, 2021 (DE) 10 2021 103 278.4

(51) Int. Cl. G16H 40/63; G16H 10/40; G16H 10/60; G16H 50/30

(52) U.S. Cl. CPC G16H 40/63; G16H 10/40; G16H 10/60; G16H 50/30

(57) ABSTRACT

A device for assisting in the intensive-care treatment of a patient for stationary use at the hospital bed of an intensive care unit is provided. The device has at least two different functional components for providing different functions for inputting and/or outputting information relating to the treatment, an input component for at least one input by a user and a dialogue component for dialogue-based interaction with the user based on the input, in order to use at least one of the provided functions. At least one of the functions is designed for filtering and outputting laboratory results of the patient.

### TECHNICAL FIELD

The invention relates to a device, a system and a method for assisting in the treatment of a patient.

### PRIOR ART

It is known from the prior art that a multiplicity of different medical devices can be used in a hospital to monitor a patient, to output information relating to the monitoring and treatment of the patient and to document this. It is likewise known that voice-controlled documentation, for example by means of voice recognition, is possible in the medical field. Different individual devices are also used when treating a patient in order to provide individual assistive functions in isolation.

### OBJECT OF THE INVENTION

The present invention is based on the object of providing the prior art with an enhancement, an improvement or an alternative.

### Achievement and Advantages of the Invention

The above object is achieved by means of the respective subject matter of the patent claims. Further features and advantageous configurations of the invention emerge from the description and the drawings. In this case, features which are described in connection with the device according to the invention also apply to the system according to the invention and/or to the method according to the invention, and vice versa in each case.

According to a first aspect of the invention, the stated object is achieved by a device for (technically) assisting in the treatment, in particular medical and preferably intensive-care treatment, of a patient, preferably for stationary use at the patient’s hospital bed, particularly preferably at the hospital bed of an intensive-care unit.

The intensive-care unit may specifically denote a ward in a hospital in which patients are subjected to intensive care using different monitoring and treatment measures and using corresponding medical apparatuses. These apparatuses comprise, for example, ventilators and/or monitoring monitors and are also referred to as external devices below for the purpose of differentiating them from the device according to the invention.

The device according to the invention and/or the external devices may each be in the form of medical devices, that is to say in particular also in the form of a medical device, for example in the sense of guideline 93/42/EWG.

The device according to the invention comprises, in particular:
at least two or at least three different functional components for providing different functions for providing, in particular inputting and/or outputting, information relating to the treatment (also referred to as information provision below), wherein the functional components are preferably designed to interchange data with one another,
at least one input component for at least one input by a user (also referred to as user input below), that is to say in particular for capturing the input by the user,
at least one dialog component for dialog-based interaction with the user on the basis of the (captured user) input in order to operate at least one of the (different) functions provided in order to preferably initiate the interchange of data by the functional components with one another and in order to particularly preferably output a result of processing of the interchanged data.

This may have the advantage that a combination of different functions is provided by a single device. The dialog component may in this case ensure simple and intuitive access to the functions. The device, for example as an assistance device, can be used to reliably assist the staff in a hospital in the treatment of the patient. For this purpose, the device according to the invention is in the form of a medical apparatus for stationary use in the hospital, for example. Furthermore, the combination of functions may have the advantage that results of the individual functions provided are combined and processed with one another as a result of the data interchange in order to output the processing result to the user.

The processing is advantageously carried out as processing of the interchanged data with one another, with the result that the data are at least partially compared with one another in order to obtain the result. For example, vital data relating to the patient are compared with the laboratory values during processing in order to filter relevant laboratory values.

Provision may also be made for at least one of the functions, and in particular a first functional component, to be designed to filter and output and/or provide laboratory values relating to the patient (also referred to as “laboratory value function” for short below). This may have the advantage that relevant laboratory values are quickly and reliably provided in an automated manner during treatment of the patient. In this case, the above-mentioned laboratory value function can be combined with further functions such as a documentation function and a knowledge database function. The device according to the invention can therefore already provide a large part of the information relevant to the treatment in a simple and convenient manner. The output of the laboratory values can be initiated in this case by the user input, for example. The user input can be carried out directly on the device on the input component or alternatively or additionally by means of an external input component, for example via a network. This makes it possible to also use the laboratory value function within the scope of telemedicine.

The first functional component may be designed, in order to provide the laboratory value function, that is to say to filter and output and/or provide laboratory values, to use further data from at least one further functional component. For this purpose, data may be interchanged between the first functional component and the at least one further functional component. In order to combine the laboratory value function with the at least one further function of the at least one further functional component, the interchanged data may be processed with one another. If the data comprise a status of the patient and/or vital parameters of the patient and/or other information relating to the patient and/or the treatment, the filtering can be carried out on the basis of these data, that is to say the currently relevant laboratory values can be chosen. For this purpose, the processing may comprise, for example, the use of a predefined set of rules and/or artificial intelligence. The output of this result of the processing, that is to say the relevant laboratory values, may improve the treatment of the patient since there is no need to manually choose the laboratory values in a complicated manner.

Assisting in the treatment is understood as meaning, in particular, machine information provision, that is to say input and/or output of the information relating to the treatment, that is to say information that is relevant to the treatment. The information may therefore be in the form of medical information such as documentation of the treatment or vital signs of the patient. In order to determine the information, the device according to the invention may have its own sensors and/or at least one interface to at least one external sensor and/or external device. The assistance in the treatment may also comprise tasks following the treatment such as documentation and provision of information relating to the (completed) treatment. It is also conceivable for the information to be provided “remotely”, such as via a network, in order to enable telemedical treatment.

The device may also be in the form of a voice assistant specifically for the medical field. In this case, the device may fundamentally be based on known voice assistants, but may be adapted in terms of the dictionary used and/or the training of a neural network for voice recognition and/or voice synthesis. The voice assistant may be used to assist the user in various tasks by means of voice inputs and/or voice outputs. For this purpose, the voice assistant comprises, for example, software which allows information to be retrieved (by means of the input component), dialogs to be conducted (by means of the dialog component) and assistance services to be provided (by means of the functional components) by means of communication in a natural, human language by virtue of the software carrying out a voice analysis for voice recognition, interpreting it semantically, logically processing it and formulating a response as the result by means of voice synthesis.

The described components and, in particular, the functional components may be in the form of a software component and/or hardware component of the device. Some of the functions may be provided, for example, only by a computer program, while other functions possibly additionally require the use of hardware (for example when capturing information using sensors). In this case, provision may be made for all described components to be exclusively part of the device according to the invention.

Although the device according to the invention can be used in a stationary manner, it can also be optionally portable, possibly even portable only by one person, in order to change the stationary place of use. For this purpose, the device has holding means such as carrying handles or the like, for example.

The dialog component is advantageously in the form of a dialog system such as a chatbot. In order to carry out the dialog with the user, the dialog component may be in the form of a computer program, for example. Interaction with the user can be carried out on the basis of the input, wherein the input is in the form of a voice and/or text and/or (also virtual) key input, for example. This means that the user inputs a notification to the dialog component via the input component, wherein this notification is then processed by the dialog system in order to output a further notification based thereon. The notifications may not exhaustively comprise:
a question (for example “What is the name of the patient” or “What is the current hemoglobin value of the patient”), in particular in the form of a notification input by the user, and/or
a response (“The name is...”), in particular in the form of a notification output by the dialog system, and/or
an item of information based on the treatment (“Document decubitus”), and/or
a request (“Request the laboratory values”), and/or
a command (“Store the documentation”).

Since both the user can input the above-mentioned notifications and the device can output the above-mentioned notifications, the input and output result in man-machine interaction which is based on a dialog between the device and the user. Such input components and dialog systems are fundamentally known, for example in the form of a voice assistant in smartphones. The notifications may also be used to retrieve information, that is to say may result in the information being output by the functions.

It may be possible for a communication path for the output by the device according to the invention to be selected on the basis of the communication path for a previous input by a dialog system. For example, the input is effected via at least one of the following communication paths:
acoustically, in particular as voice,
visually, in particular as text.

It may be possible to use exactly the same communication path for the output as for the input. Furthermore, the communication path may also vary for different inputs, with the result that a voice output is also then selected in the case of a text input and a text output is also then selected in the case of a voice input.

The user preferably differs from the patient and is, in particular, a medical professional such as a doctor or a nurse. For example, provision may be made for only a particular group of persons (such as the medical professionals in a hospital) to be authorized to use the device. Authentication of the user on the device may be provided for this purpose, for example. The device carries out the authentication itself, in particular, for example on the basis of an input such as a code and/or on the basis of voice recognition by means of the input component or the like.

In order to carry out the dialog-based interaction, the dialog component may have software which allows information to be retrieved from the user and/or dialogs to be conducted and/or the provided functions to be provided in the form of assistance services by means of communication in a spoken and/or written, natural human language (that is to say acoustically and/or in a text-based manner). For this purpose, the software can carry out a voice analysis for voice recognition, can interpret it semantically and/or logically process it and can formulate a response on the basis of the processing as the result by means of text or acoustic voice synthesis. If the input relates to a request for the laboratory values, for example, the laboratory values can be output as an acoustic voice output and/or visually as the response. If, in contrast, the input relates to documentation, for example, this can be stored as information. The information can therefore be input by voice by means of the input by the user and the information can be output by voice by means of the output.

It may be possible for the functional components of the device according to the invention to at least partially access a common database. The database may be stored in an internal memory of the device and/or outside the device, for example in an information system, or may be provided by an external device such as a monitoring monitor. In this case, the device according to the invention may have a data interface to the external database. The database comprises, in particular, patient information, that is to say patient data such as laboratory values or monitored vital parameters of the patient.

For output, the device according to the invention may advantageously have at least one output component such as a screen and/or a loudspeaker and/or another display element. The input component may also at least partially have the output component if the input component is in the form of a touchscreen or comprises a touchscreen, for example.

In addition, it may be possible for the device according to the invention to comprise at least one electronic interface to external output components. This makes it possible to flexibly expand the device according to the invention. The interface may be, for example, an interface to an emergency call system such as a nurse call system of the hospital, in order to output an alarm in an emergency. The emergency may be automatically detected, for example, by a further functional component of the device according to the invention on the basis of an analysis of the patient data and the alarm may then be initiated. Telemedical assistance may also be enabled in this manner in order to transmit an audio and/or video recording to an external output component via a network, for example.

According to a further aspect of the invention, the invention likewise relates to a system comprising:
a device according to the invention,
an information system in order to at least partially provide the information for storage and/or reading-out during input and/or output.

According to a further aspect of the invention, a method for (technically) assisting in the treatment, in particular intensive-care treatment, of a patient is also protected. In a method according to the invention, the following steps may be carried out, preferably in succession in the stated order, wherein the steps can also be carried out repeatedly individually and/or collectively:
providing a device, in particular a device according to the invention, preferably for stationary use at the hospital bed of an intensive-care unit,
providing different functions for inputting and/or outputting information relating to the treatment by means of the device,
capturing an input by a user by means of the device, in particular by means of an input component,
initiating dialog-based interaction with the user by means of the device on the basis of the captured input in order to operate at least one of the functions provided, in particular by means of a dialog component.

In this case, at least one of the functions provided may optionally filter and output laboratory values relating to the patient.

The same advantages as described in connection with a device and/or method according to the invention can therefore arise for the method according to the invention.

Initiating, in particular carrying out, the dialog-based interaction is understood as meaning, in particular, the fact that the device, and specifically the dialog component, captures the input by the user using the input component, evaluates said input and outputs a response based thereon. The evaluation can be trained in advance, for example, using an algorithm such as a neural network or another machine-trained algorithm, and/or can be carried out according to predefined rules.

### Advantageous Configurations of the Invention

Furthermore, according to a further advantage, it is possible in the device according to the invention for at least one of the following functional components to be provided, wherein the stated numbering is used only for unambiguous designation and does not imply the number or order of the functional components in the device:
a first functional component for providing the function for filtering and outputting the laboratory values relating to the patient as a first function (also referred to as “laboratory value function” for short below),
a second functional component for providing a second function for documenting the treatment (also referred to as “documentation function” for short below),
a third functional component for providing a third function for retrieving information from a medical knowledge database (also referred to as “knowledge function” for short below), in particular for responding to medical questions,
a fourth functional component for predicting a medical condition of the patient (also referred to as “prediction function” for short below), in particular for predicting a critical (that is to say life-threatening) condition of the patient, and/or for outputting a proposed treatment on the basis of an analysis of patient information,
a fifth functional component for assisting and/or guiding a medical professional and/or for documentation during treatment according to a medical guideline (also referred to as “guideline function” for short below),
a sixth functional component for using checklists during treatment (also referred to as “checklist function” for short below),
a seventh functional component for at least partially combining the input and/or output information of the provided functions (also referred to as “combining function” for short below),
an eighth functional component for providing a reminder to document the treatment,
a ninth functional component for automatic documentation,
a tenth functional component for telemedical assistance during treatment,
an eleventh functional component, in particular also referred to as a connection component, for determining and providing at least one item of patient information and/or for operating and/or controlling and/or automating at least one external function, in particular a treatment function, preferably of at least one external device.

For input and/or output and/or in order to use the user input, the functional components may at least partially use the input and/or dialog and/or output component of the device according to the invention, but alternatively or additionally may also use external input and/or output components, for example via a network. This enables use in telemedicine, for example.

In the case of the laboratory value function, the output information relating to the treatment may be the laboratory values which are determined, for example, from an information system, in particular a data network of the hospital. For this purpose, the device may have a corresponding interface to the information system or data network.

In the case of the documentation function, the input and/or output information may be documentation text which is stored in the device itself or in the information system.

Furthermore, it may be possible for the documentation function to carry out documentation by scanning in at least one document (such as a text document or photo), in particular by means of the device according to the invention, and digitally storing it. For this purpose, the device according to the invention may have a scanner and/or a camera. The input information may therefore be the document which is stored in the device itself or in the information system. It is then possible for the document which has been scanned in to be processed in order to make it possible to automatically extract information from the document. This processing can be carried out by the device according to the invention and may comprise text recognition (OCR), for example. The documentation relating to the patient can then possibly be filled out automatically, such as the filling-out of an admission form on the basis of this information.

In the case of the knowledge function, the information stored in the knowledge database may be the output information of the function, wherein the knowledge database is stored in the device itself, for example.

In the case of the prediction function, an item of information relating to a predicted future condition of the patient may be the output information. The prediction is made in this case, for example, by means of a neural network or an algorithm, in particular a machine-trained algorithm, which predicts the condition on the basis of the available data (such as vital parameters and/or laboratory values) relating to the patient and/or the treatment. Such prediction systems are fundamentally known, wherein a special feature according to the invention may be the fact that such a system is integrated with the further functions in the device. This makes it possible to be able to directly use the information output by the other functions for the prediction and therefore to provide a central location for processing and outputting all information. The data relating to the patient and/or the treatment are provided as patient information, for example. Patient information can be provided, for example, by means of an input on the device according to the invention and/or by reading it from an information system by means of an interface of the device for transmitting data and/or by capturing it by means of an optical and/or acoustic interface of the device and/or by means of an eleventh functional component of the device. It may also be possible for at least one step for the treatment to be automatically initiated on the basis of the prediction. For this purpose, the eleventh functional component, for example, may automatically control at least one external device. The at least one step may be prestored, for example, in a set of rules, for example particular control of the external device for particular predictions.

In the case of the guideline function, the treatment steps predefined by the guideline may be the output information and, in the case of the checklist function, the checklist items may be the output information. The user may then possibly input completion of these steps or items, which can be documented by the respective function.

The documentation function and/or the checklist function may query predefined documentation and/or checklist items, for example. The documentation and/or checklist items may each comprise at least one predefined keyword (for example “batch number”) or input field (for example for a text input or for “ticking off”). Furthermore, the predefined documentation and/or checklist items may be assigned to a treatment step such as the administration of units of blood. For example, the documentation and/or checklist items are the batch number and/or the time and/or the reason for the treatment step. The documentation and/or checklist items may be queried, for example, by capturing an input for an input field for each documentation or checklist item. The integrity and/or completeness of the inputs may then be checked. For example, a check is carried out in order to determine whether all relevant documentation and/or checklist items have been input. In this case, the relevant documentation and/or checklist items are predefined, in particular, and may also depend on the current patient’s condition and/or the current treatment. The relevant documentation and/or checklist items may be determined in an automated manner in this case on the basis of the information or data from further functional components in accordance with this dependence. The documentation and/or checklist items can also advantageously be at least partially automatically filled out on the basis of the information or data from other functional components. A suggestion for documentation and/or checklist items may likewise be output automatically, in particular on the basis of the information or data from other functional components. For example, the normal range of the laboratory values and/or the drugs provided for the treatment can be automatically displayed for checking.

In the case of the combining function, some of the above information may be output in a combined form (for example on a screen). The selection of the information for combining may be predefined, for example, or may also be dynamically generated (for example by means of an algorithm and/or a neural network) on the basis of an analysis of the current patient status.

A fundamental advantage of combining the various functions may be that the functional components can interchange information, that is to say data, with one another and/or that the input and output of the information for various ones of the functions is pooled at a central location.

In addition, it may be possible within the scope of the invention for the device to be (partially or completely) in the form of a stationary, bedside device, preferably in order for the user to carry out the input in a manner adjacent to the hospital bed of the patient. In other words, the device may be in the form of a stationary apparatus which is installed in the vicinity of the hospital bed in order to provide the functions in a patient-related manner. Such a device may also be referred to as a bedside device. For the stationary design, the device may have at least one stand means and/or stabilization means such as a supporting surface for the hospital floor or a support structure such as a stand. In this case, the stand means may have a movement means such as a roller or a wheel in order to also move and thus position the device for stationary use at another location. The stand means may also be designed for the immovable, fixed installation of the device or may have a fixing means for fixing the movement means in order to block a movement of the device after positioning.

It is also conceivable for some of the functions and/or structural parts of the device and/or system according to the invention to not be arranged bedside, that is to say for them to also be able to be provided outside (for example outside the patient’s room).

According to one advantageous development of the device according to the invention, the device may have at least one (in particular electronic) interface to at least one information system, in particular a patient data management system (PDMS for short), preferably an intensive information management system of the intensive-care unit, in order to store and/or read out the information at least partially via the information system during input and/or output. The interface comprises, for example, an electrical plug or a socket in order to transmit electrical signals for interchanging information to the information system. Alternatively or additionally, it is possible for the device according to the invention to itself (internally) have and provide its own information system, in particular a PDMS. It is therefore possible to also be able to use the information system when there is no external information system. It is then also conceivable for the device according to the invention to have an interface for external devices to this internal information system so that further external devices can access said information system and can retrieve information therefrom.

The information system may be in the form of a hospital information system for managing treatment-relevant information relating to the patient, in particular current and/or historical laboratory values. The information system may also be designed to at least partially output the information in the form of patient information.

It is also possible for the device to have at least one interface to an information system and/or to at least one external sensor and/or to at least one external device in order to provide patient information relating to a condition of the patient. The patient information comprises, for example, vital parameters (also referred to as vital signs or vital data) which are determined by the external sensor such as a blood pressure monitor. The patient information may also comprise data relating to the age, illnesses and/or laboratory values of the patient, which are provided by the information system.

It is also conceivable for the input component to be designed to recognize a voice of the user in order to receive the input by voice. In other words, the input component may be in the form of a voice recognition apparatus. Alternatively or additionally, the input component may be in the form of a human interface device such as a keyboard or a touchscreen. The voice recognition apparatus may have, for example, a microphone and/or an electronic arrangement for subjecting the microphone signal to analog/digital conversion and/or voice recognition software. The software may have an (evaluation) means trained by machine learning, in particular a neural network or another machine-trained algorithm, for voice recognition.

Within the scope of this invention, a neural network is understood as meaning, in particular, an artificial neural network, that is to say an algorithm which can be trained or formed by training data. For this purpose, a weighting which is specific to the function of the network can be formed during training.

Furthermore, the input component may also optionally enable a user input using gestures. For this purpose, an optical interface, such as a 360° camera, of the device according to the invention may be evaluated, for example, in order to determine the gestures. The functional components, for example, can be controlled on the basis of the gestures.

Provision may also be made for the dialog component to be designed to evaluate the input by means of a machine-trained evaluation means, in particular a neural network. In this case, the recognized voice, for example, can be used as an input for the evaluation means in order to use the output of the evaluation means as an assignment of the input to a predefined notification. In other words, the evaluation means may be trained to classify the input with respect to predefined notifications. In this manner, the user can input a notification to the dialog component via the input component. The dialog component can then call one of the functions, for example, on the basis of this notification. Furthermore, the dialog component may assign the notifications to the functions, wherein the assignment may be predefined for various notifications. This technical implementation of such an assignment to notifications is known in other fields in conventional voice assistants. For example, for the voice input “What will the weather be like”, known voice assistants can assign this input to the corresponding notification which results in the activation of the function “weather report”. However, use in the field of medical treatment has considerable advantages in daily clinical practice since this type of man-machine interaction is particularly suitable for the situation in an intensive-care unit, for example. It is therefore possible to rely on an intuitive request for the information and to simplify operation by virtue of the dialog-based interaction. For example, the dialog component also makes it possible in this case for a query to be output in the case of unclear notifications from the user, in order to request the user to specify the previous input.

Provision is advantageously made for the function for filtering and outputting laboratory values relating to the patient to be designed to carry out the filtering on the basis of patient information, in particular in order to automatically filter the laboratory values according to relevance to the treatment. In other words, the filtering in this case chooses and possibly outputs those laboratory values which are classified as relevant by the function. For this purpose, provision may be made for the function to create a data fingerprint for the patient. The data fingerprint is created, for example, on the basis of the available patient information, in particular such that the data fingerprint is then unique for the specific combination of values of the patient information. The data fingerprint is therefore also specific to and preferably unique for the condition of the patient. The function may then use artificial intelligence and/or a predefined set of rules to assign the data fingerprint, and therefore also the condition of the patient, to a selection of laboratory values which are classified as relevant to the treatment from then on. The relevant laboratory values can then be output by voice or visually.

The patient information comprises, for example, laboratory values from a laboratory test, in particular blood sampling from the patient, and/or a blood pressure of the patient and/or vital data relating to the patient and/or information relating to the age and/or previous illnesses and/or drug administration and/or test results for the patient. This patient information may possibly be partially directly determined by one of the functional components and may partially also be read from an information system.

It is possible for the data fingerprint to be formed from the patient information by processing and/or comparing the patient information. This data fingerprint can then be assigned to the relevant laboratory values by means of a set of rules and/or by means of an algorithm such as a neural network or another machine-trained algorithm. In this manner, the data fingerprint can be taken as a basis for determining which of the available laboratory values is currently relevant. The relevant laboratory values can then be directly output or (only) the laboratory values which are not in the normal range can be output. In this case, the set of rules may comprise a simple assignment (for example considering a particular laboratory value to be relevant in the case of a particular value of the vital data).

The patient information comprises, for example, at least one of the following items of information:
current vital signs of the patient,
a historical progression of vital signs of the patient,
current laboratory values and/or a historical progression of laboratory values relating to the patient,
information relating to illnesses of the patient,
information relating to at least one biometric feature of the patient, for example body weight or height,
information relating to at least one treatment for the patient.

The patient information may be at least partially used as an input for an evaluation means such as a neural network in order to use an output of the evaluation means as a list of the laboratory parameters (such as leucocytes or hemoglobin or the like). This list may indicate those laboratory parameters which should be output by the laboratory value function as information. The evaluation means therefore carries out filtering from all available laboratory parameters. The evaluation means may be accordingly interpreted as a filter which filters the laboratory values, which are intended to be output, on the basis of the patient information.

Accordingly, it is likewise possible for the filtering function, that is to say the laboratory value function, and/or the first functional component to have a machine-trained evaluation means such as a neural network. In this case, the neural network is trained, for example, by means of at least one of the following steps:
providing at least one item of input information or a multiplicity of items of input information, each having predefined patient information, wherein the respective input information comprises, for example, a combination of the patient information, for example a combination of specific values for the vital signs and/or the illnesses and/or for historical laboratory values,
providing an item of predefined output information for each of the items of input information which are specific to a desired filtering result, for example the specific list of the laboratory values which are intended to be output when the corresponding input information is present,
respectively using the input information as an input for the evaluation means and respectively using the predefined output information as a learning specification (for example a ground truth),
carrying out the machine training of the evaluation means, for example in the form of supervised learning,
providing the trained evaluation means in the device according to the invention, for example by storing the weighted neurons resulting from the training and/or a classifier resulting from the training in the device.

The available patient information can then be used as an input for the trained evaluation means provided and the output of the evaluation means can be used as the laboratory values to be output.

In the device, provision is also optionally made for the filtering function, that is to say the laboratory value function, and/or the first functional component to have an evaluation means which has an (in particular predefined) set of rules for assigning combinations of the patient information to a selection of the laboratory values in order to output the selected laboratory values to the user. For this purpose, the evaluation means comprises, for example, a corresponding (possibly manually) predefined assignment, for example in the form of a table. Alternatively or additionally, the evaluation means may be in the form of a computer program which assigns particular combinations of patient information to particular combinations of laboratory parameters according to the set of rules. In order to optionally reduce the complicated preparation of the evaluation means in this case, the evaluation means may also be machine-trained as an alternative or in addition to generating the set of rules. In this case, the set of rules may be interpreted as a weighting of neurons or a classifier of the evaluation means.

Provision may be made for the evaluation means for filtering to be designed to detect, in particular predict, a future deviation of at least one laboratory value from a normal range in order to preferably output this laboratory value. The first functional component or the first function, and in particular the evaluation means, can therefore be designed to detect a future deviation of at least one laboratory value from a normal range and/or to predict which laboratory values will be outside a normal range in future. This makes it possible for those laboratory values for which the prediction determines a future deviation from the normal range to be filtered and output by the function. For this purpose, the evaluation means may have an accordingly trained neural network. One possibility for this training is described above. This makes it possible to present relevant laboratory values in a particularly clear manner.

The future deviation of a laboratory value may be determined, for example, by analyzing different laboratory values for a sample, in particular a blood sample. Different laboratory values are, for example, different analytical results which can be obtained from the sample. Laboratory values or analytical results are, for example, the number of red blood cells, the number of white blood cells, the number of blood platelets, the iron content, etc. Additionally or alternatively, the future deviation may also be determined by analyzing a laboratory value over different samples which have been taken in temporal succession. A joint consideration of a temporal development of different laboratory values may likewise be characteristic of the future deviation of a laboratory value.

According to a further optional development of the device according to the invention, one (a fourth) of the functional components may be provided for the purpose of providing a (fourth) function for predicting a medical condition of the patient in order to predict a disease progression of the patient on the basis of patient information. An evaluation means of the (fourth) functional component may likewise be provided for this purpose. This evaluation means comprises, for example, a set of rules which comprises an assignment of combinations of the patient information to a predicted condition of the patient. For example, the disease progression may be determined in this manner in the form of an improvement or deterioration in the patient’s condition. As previously described for the laboratory value function, the set of rules may be manually predefined or machine-trained. It is possible for a critical condition to be predicted when particular combinations of the patient information are present. The prediction of the critical condition may result in an alarm being output in the device. An alarm function for outputting a visual and/or acoustic alarm may be provided for this purpose.

In addition, one (in particular the fourth) of the functional components may be provided for the purpose of providing a proposed treatment on the basis of an analysis of patient information. For the analysis, the functional component may have an evaluation means which comprises, for example, a neural network or a manually predefined set of rules. As an input, the evaluation means may use at least one item of patient information, for example a combination of at least laboratory values and/or a progression of laboratory values and/or vital signs and/or patient data such as age and/or previous illnesses and/or the like. The output of the evaluation means may be used as the proposed treatment. In this case, it may be possible for the proposed treatment to comprise settings for a ventilator. In order to design the evaluation means, the latter may be manually and empirically configured, for example, in such a manner that it has an assignment of particular combinations to particular proposed treatments. The evaluation means may also possibly be machine-trained, for example by means of reinforcement learning and/or so-called “evolutionary reinforcement learning”. It is therefore possible for this function of providing the proposed treatment to be able to be improved further during operation. In other words, the evaluation means can also be adapted further after the device according to the invention has been started up, in particular on the basis of inputs by the user and/or on the basis of the further development of the patient information. In this learning method, the patient information may therefore influence the benefit function or reward function. Improved learning can therefore be carried out in order to be able to reliably output proposed treatments for the user.

In addition, one (a fifth) of the functional components may be provided for the purpose of providing a (fifth) function for assisting and guiding a medical professional and/or for documentation during treatment, in particular according to at least one medical guideline. The fifth function may output, for example, instructions from the guideline, preferably in the form of a voice output. The guideline may be prestored in the device and/or may be determined from the information system via the interface. In this case, the guideline may have the instructions which are to be output and are possibly predefined as a sequence. The performance of the instruction may then be confirmed by the user, possibly via the input component, for example by voice by means of a corresponding acoustic notification or a confirmation word. In the device, the confirmation may automatically result in the performance of the instruction being documented. This documentation can then be stored for subsequent use in the device and/or in the information system. This makes it possible to reliably and securely document the treatment.

It is also conceivable for the documentation to be carried out by providing a dictation function for the user. For example, the user can request the activation of the documentation function via the input component. The dictation function can then be activated so that subsequently spoken text is converted into written text for the documentation and is stored. Alternatively or additionally, the documentation can be carried out by scanning in a document and automatically extracting the information therefrom. The documentation function described above is used for this purpose, for example.

It is also conceivable for information stored in the device to be automatically transmitted to the information system via the interface in order to be assigned to a patient file, for example. This makes it possible, for example, to assign the previously dictated text to the documentation in the patient file.

According to a further optional improvement, one (a sixth) of the functional components may be provided for the purpose of providing and automatically confirming at least one checklist for the treatment. For this purpose, the at least one checklist may be stored in the device and/or in the information system, for example, and may be retrieved there following a corresponding request from the user. Furthermore, confirmation of the checklist may also be automatically (without manual assistance) effected and stored in the background if the presence of prerequisites provided for this purpose is identified on the basis of the patient information. For example, one prerequisite in this case is for a particular treatment to be carried out, wherein the arrangement of this treatment is identified on the basis of the patient information, in particular documentation of the treatment. This makes it possible to maintain the checklists for the treatment in a more reliable manner.

It is also advantageous if at least one of the functional components is designed, in order to provide at least one of the functions, to automatically and in particular repeatedly carry out processing for the purpose of determining the information (relating to the treatment) in order to preferably provide the determined information for subsequent retrieval by the input component and/or for subsequent performance of the function. Accordingly, this processing and therefore the provision of the information can be carried out in the background without a specific request and/or manual activation of the function being present for this purpose. This avoids the situation in which a user first has to wait for several seconds or minutes for the output of the information following a request. For example, the automatic processing takes place in an interval of time of at most 1 min (minute) or at most 10 min or at most 1 h (hour) in order to always be able to provide current information for the subsequent request. If the subsequent request is made within this interval of time, only the previously provided information is therefore output and there is no wait for the completion of further processing for the output. An electronic timer component, for example, may be provided for the purpose of initiating the repeated processing, that is to say in particular the performance of the function. The function also carries out this processing in a manually initiated manner and otherwise automatically in the background, for example, in order to provide the information relating to the treatment and to input and/or output it if necessary. In this manner, the function, for example the laboratory value function, can determine the information, such as the laboratory values to be output, on the basis of the input even before this function is operated.

Provision may be made for automatic processing (in the background) to comprise execution of at least one of the evaluation means of the device which are provided within the scope of the invention. For example, the evaluation means of the first functional component can be repeatedly executed in the background in order to carry out the computation-intensive prediction of which of the laboratory values will be outside a normal range in future. The prediction of the medical condition may also be repeatedly automatically carried out in the background by the fourth function. Execution in the background is therefore provided, in particular, for those algorithms which require a longer processing time.

It is likewise advantageous if the dialog component is designed to detect a wake-up signal in order to initiate a dialog for operating the functions when the wake-up signal is detected. The wake-up signal is, for example, a particular predefined notification input by the user, such as a voice notification or a particular spoken word. Provision may be made for the functions to be able to be operated by means of the dialog component or by means of the input only when the wake-up signal has been previously detected. For example, the dialog is initiated after the wake-up signal has been detected. If a further input is then not made within a predefined period, for example a maximum of 5 or 10 s, the dialog can be deactivated again. For example, this results in the device changing into an energy-saving mode until the wake-up signal is detected again.

It is likewise possible for one (an eighth) of the functional components to be designed to provide a reminder to document the treatment. For example, it may be specified that certain processes must be regularly documented during treatment. On the basis of the database, the functional component can check whether part of the documentation is missing. This check is carried out, for example, at predefined and/or configurable intervals of time. If the check reveals absence of the documentation, a corresponding reminder can be acoustically and/or visually output.

It is also conceivable for the (eighth) functional component to provide a control function for the documentation. In this respect, it is possible to provide a reminder if details are still missing in the documentation, for example. This “control function” may carry out, for example, a comparison with a prestored template which indicates which details of the documentation must be included. It is therefore qualitatively ensured that the necessary data are acquired during treatment.

It is also conceivable for one (a ninth) of the functional components to be designed to automatically document the treatment on the basis of an analysis of an item of user information that has been input. For example, it is conceivable for the user information that has been input to comprise (for example dictated) free text which is not suitable as documentation on the basis of predefined criteria and/or categories. The user information is, for example, an input by the user in voice form and/or in text form and may comprise, in terms of content, documentation contents relating to the treatment. That part of the user information which is suitable for documentation can then be identified on the basis of the analysis. Alternatively or additionally, it is conceivable for certain processes to have already been documented at another point. So that these do not have to be manually documented several times, the functional component can automatically document such processes at least one further time. Automatic documentation may possibly also use the documentation function, for example in order to scan in a document and to automatically extract the information which is documented therefrom.

It is likewise possible for an optical interface of the device according to the invention and/or of the system according to the invention and/or as an independent apparatus to be provided in order to optically determine at least one item of patient information in an environment of the device. If the optical interface is part of the device according to the invention, it can be permanently integrated in a housing of the device or can be alternatively connected to the further components of the device via a connection means such as a cable. The cable may also be used to electronically interchange data between the interface and at least one of the functional components and/or to supply energy to the interface. The optical interface has the special feature that the patient information is not determined by means of digital data transmission, but rather is optically captured. This is necessary, for example, when an external device, such as an external sensor, does not provide an electrical interface. This makes it possible to determine patient information, such as vital signs of the patient, even from those external devices for which no dedicated device interface is provided. In other words, the patient information may come from the external device, in particular the display of the device, that is to say may be optically “read” from the display by the interface, for example. For this purpose, the optical interface is, for example, a camera which may be aligned with the display of the external device in order to repeatedly record it. The external device is, for example, a patient monitor for monitoring vital parameters.

According to one advantageous development, the optical interface may have at least one image sensor in order to optically capture and in particular monitor at least one external device, in particular a medical device, preferably a monitoring monitor, preferably of the intensive-care unit, in order to determine the patient information on the basis of the optical capture. The optical interface may therefore capture an optical representation of the patient information which is originally output by the external device for visual perception by the user. The patient information can then be provided from this visual information in digital form by the optical interface. For this purpose, the optical interface optionally has its own text recognition module in order to recognize text captured by the optical capture and to convert it into data that can be digitally processed. Alternatively, a processing component of the device according to the invention, such as a data processing system, that is to say a computer or a microcontroller for example, can also carry out this text recognition.

It is also conceivable for the optical interface to have at least one camera in order to record at least one screen of the external device or of the monitoring monitor in order to determine the patient information on the basis of an evaluation of the optical recording. As described above, the evaluation comprises text recognition, for example. In this manner, the patient information such as vital signs (for example blood pressure and pulse) can be determined in a flexible and reliable manner.

It is also possible for the optical interface to have at least one or precisely one omnidirectional camera, that is to say a 360° camera, in order to capture a plurality of external devices. A complete image of the environment can therefore be used to capture the patient information. In addition to the above-mentioned “reading” of the patient information from external devices, this also makes it possible to evaluate other circumstances, such as the presence or absence of the patient or user. This information from this evaluation may likewise be communicated to further functional components.

In the device and/or method and/or according to the invention, it is also possible for at least one of the functions provided to comprise a telemedicine function. The telemedicine function is provided, in particular, by a (tenth) functional component and may be in the form of a technical function for telemedical assistance. For this purpose, the telemedicine function may comprise, for example, a videoconferencing function and/or an audio transmission function and/or information provision via a network. Accordingly, the device according to the invention may be designed to technically at least partially provide telemedicine.

It may also be possible for a or the optical interface of the device according to the invention, which has, in particular, at least one camera, preferably an omnidirectional camera (360° camera), to be used for telemedical assistance. Accordingly, the optical interface may also be used by a (tenth) functional component to provide the telemedical assistance.

Telemedical assistance is understood as meaning, in particular, the fact that the device provides technical aids for telemedicine in order to at least partially technically enable telemedicine. For this purpose, the device may provide, for example, video transmission and/or a videoconference and/or sound transmission. It is also possible for the information relating to the treatment or the patient information to also be at least partially provided in addition to audio and video information. In this context, reference may be made to the fact that the information provision described above is effected telemedically.

It is conceivable for the information relating to the treatment to also be at least partially input and/or output via a network. In this case, as an alternative or in addition to using an input and/or output component of the device according to the invention, it is also possible to use an input and/or output component outside the device according to the invention for input and/or output. The input and/or output component outside the device according to the invention is, for example, part of an external device such as a computer outside the intensive-care unit or in a doctor’s room.

The device according to the invention may possibly provide a web interface for input and output, for example for user input and/or for information provision. The input and/or output component outside the device according to the invention may be designed, for example, to provide the user with the web interface. Information from the information system can also be provided via this web interface, for example.

For telemedical assistance, the device according to the invention may have a (tenth) functional component which has an interface to at least one network, in particular a communication network. The network comprises, for example, a mobile radio network, for example according to 5G, and/or a data network and/or the Internet. Accordingly, the interface may be in the form of a network interface and/or a mobile radio interface (such as a mobile radio module). It is therefore possible to transmit information such as the information relating to the treatment or patient information and/or inputs and outputs of the functional components and/or user inputs and/or audio and/or video information via the network. The information or audio and/or video information may be determined or recorded locally at the device according to the invention, for example, and may be transmitted to an external device via the network. The external device is, for example, a user’s computer further away from the patient, for example outside the intensive-care unit or in a doctor’s room.

The device according to the invention may likewise have a light module in order to control lighting for the patient. For example, the lighting may comprise background lighting in the patient’s room. The light module may be in the form of a further functional component and may possibly have an interface to the external lighting. The lighting is controlled on the basis of the user input, for example.

It may also be advantageous if an acoustic interface of the device according to the invention and/or of the system according to the invention and/or as an independent apparatus is provided in order to acoustically determine at least one item of patient information. Similarly to the reading of the patient information, this may enable acoustic perception of the patient information. However, unlike in the input component, the perception of a voice for input may not be meant here, but rather the acoustic perception of (in particular artificial) signals and/or sounds and/or ambient noise. For example, warning sounds may be captured in order to infer a condition of the patient on the basis of this patient information by means of a further functional component. The acoustic output of a vital signs monitor may also be monitored. The vital signs monitor outputs the heartbeat in the form of an acoustic signal, for example: one signal for each heartbeat. The heart rate can therefore be determined as patient information by monitoring the repetition rate.

Like the functional components and/or the input component and/or the dialog component, the acoustic and/or optical interface may also be part of the device according to the invention. The device may therefore be in the form of an autonomous apparatus having its own energy supply and data processing system. In order to nevertheless make it possible to interchange information with external systems, at least one interface may be provided.

In a system according to the invention, it is possible for the optical and/or acoustic interface to be in the form of an external apparatus and to therefore not be part of the device according to the invention.

Provision may also be made for the input component to be designed, in order to carry out beamforming, to determine a direction of the user as an acoustic input on the basis of the input and/or to improve a voice input by means of the beamforming. The beamforming can be used to attempt to extract a sound source and therefore the user in a room. It may previously be necessary to determine the direction of the sound source. For this purpose, the input component, for example, can determine an angle of incidence of the sound waves in order to estimate the direction of the user. It is also possible to determine the directions of a plurality of users in this manner, which can then be individually extracted. This makes it possible to considerably improve the voice recognition. For this purpose, the input component may also have an evaluation means such as a neural network. The determination of the direction of the user or of the sound source is referred to as direction recognition for short below.

It is likewise conceivable for the dialog component to be designed to detect a or the wake-up signal described above in order to initiate a dialog for operating the functions when the wake-up signal is detected.

In order to capture the input and/or the wake-up signal, it may be possible to provide and/or use at least two or at least four or at least six or at least eight or precisely eight microphones of the device according to the invention. The microphones may be oriented in different directions starting from the device according to the invention. It is therefore possible to carry out direction recognition or the beamforming by comparing the microphone signals from the microphones. It may be possible for the direction to be recognized by means of triangulation on the basis of the different propagation times of the microphone signals, that is to say the sound waves which have arrived.

After the wake-up signal has been detected, the beamforming or the direction recognition can be advantageously already carried out on the basis of the wake-up signal in order to determine the direction of the user and/or to improve the voice input. The detection of the wake-up signal may possibly trigger the direction recognition or the beamforming. The wake-up signal is advantageously initially detected on the basis of the microphone signals from the microphones. These microphone signals can then already be compared with one another in order to directly carry out the direction recognition or the beamforming on the basis of the wake-up signal. This has the advantage that, after the wake-up signal has been detected, no further acoustic input is needed in order to be able to carry out the direction recognition. The direction recognition may also be directly used for the further acoustic inputs in order to improve the recognition accuracy.

It may also be possible for the detection of the wake-up signal to comprise processing in order to reduce the ambient noise from the microphone signals. For this purpose, bandpass filtering or algorithmic noise suppression or the like is used, for example. The detection of the wake-up signal may likewise comprise pattern recognition in order to identify a predefined acoustic pattern in the wake-up signal. The predefined pattern comprises, for example, a particular word sequence such as “Hi Mona” or the like.

It is also conceivable for the dialog component to be designed to control at least one or more of the functional components on the basis of the input in order to operate the functions provided. For example, it is conceivable for particular functional components to be controlled on the basis of predefined words or voice patterns. It is also conceivable for the control to be carried out on the basis of a predefined set of rules and for it also to be dependent on the condition of the patient or the current treatment. The patient’s condition or the current treatment can be determined by the device according to the invention, possibly by means of previous inputs, and/or by the functions of the functional components.

It is also advantageous if the device according to the invention is modular in order to subsequently supplement further functional components, in particular. Hardware and/or software components in the device, for example, may be supplemented and/or activated for this purpose. This makes it possible to easily and flexibly expand functions, in particular by at least one of the functional components. For this purpose, the device according to the invention may have corresponding interfaces for further components.

It is also possible within the scope of the invention for the functional components and/or the input component and/or the dialog component to each be at least partially in the form of electronic components of the device.

At least one computer program, in particular a computer program product, may be provided for the purpose of carrying out the steps of a method according to the invention. In this case, the at least one computer program may comprise instructions which, when executed by at least one processing apparatus, cause the latter to carry out the steps of the method according to the invention. The at least one processing apparatus may be part of the device and comprises, for example, a computer and/or at least one microcontroller and/or at least one processor and/or at least one integrated circuit. Furthermore, the processing apparatus may also comprise the at least one processor as a digital signal processor and/or a graphics processor and/or may be designed for parallel execution of the instructions. The computer program may be stored in a non-volatile memory of the device and may be read out by the processor.

The device according to the invention may have an (in particular eleventh) functional component which comprises at least one optical and/or acoustic interface and/or is connected to such an interface, possibly also via radio, for the purpose of transmitting data in order to optically and/or acoustically determine at least one item of patient information in an environment of the device according to the invention. The determined patient information can then be provided for the device according to the invention and preferably for further functional components. Provision is effected, for example, by storing the patient information in a non-volatile manner in a data memory of the device according to the invention.

It may also be possible for a or the eleventh functional component to be designed to operate and/or control and/or automate and/or receive information from at least one external function, in particular a treatment function, preferably of at least one or various external device(s). At least one and/or the at least one optical and/or acoustic interface of the device according to the invention may be used for this purpose. In other words, operation and/or control and/or automation and/or information reception can be carried out by interchanging data with the external device via the interface.

It may be possible for one or more different external devices to be present in the intensive-care unit, for example a syringe pump and/or a patient bed and/or a sonography device and/or at least one lighting means. The respective function of the device may comprise, for example, adjusting the bed and/or configuring the syringe pump and/or receiving sonography images and/or adjusting the lighting. The interface may be in the form of an electronic interface, for example a ZigBee or Message Queuing Telemetry Transport (MQTT) interface.

It may be possible for the operation and/or control and/or automation and/or information reception to be initiated by the dialog system on the basis of the input on the device according to the invention. According to a first possibility, provision is therefore made for the operation and/or control and/or automation and/or information reception to be triggered by the dialog system. This has the advantage that the input and output methods of the device according to the invention can be used to operate the external devices. This makes it possible to initiate, for example, a wake-up phase on the basis of the input, for example, by accordingly adjusting the lighting means. Information relating to the treatment, such as sonography images, may also be made available to other functional components as a result of the information being received.

According to a second possibility, provision may be made for the operation and/or control and/or automation and/or information reception to be triggered by a further functional component. For example, the operation and/or control and/or automation and/or information reception may be initiated on the basis of the output of the alarm by the alarm function and/or a prediction by the fourth functional component and/or on the basis of an automation specification such as the time. The automation can be carried out, for example, according to a prestored specification, for example a particular lighting specification for the lighting means. It is also possible, in this manner, to incorporate further external devices in the system according to the invention in order to read out patient information therefrom.

The functional components described within the scope of this invention may be at least partially in the form of software components and/or hardware components of the device according to the invention, that is to say the functions of the functional components may also be at least partially provided by a computer program. The computer program or the functional component uses, for example, an interface to the data memory in order to read out the patient information provided.

In accordance with a method according to the invention, provision is advantageously made for the following steps to be carried out, in order to optically and/or acoustically determine at least one item of patient information and to provide the at least one item of patient information, in particular subsequently, preferably by means of the (in particular eleventh) functional component and/or an optical and/or acoustic interface and/or an electronic data processing system such as a server:
optically and/or acoustically capturing at least one external medical device, also referred to as an external device for short below, in order to obtain, by means of the capture, at least one item of capture information, in particular at least one item of optical information and/or at least one item of image information and/or at least one item of acoustic information,
processing the capture information obtained, wherein the processing preferably comprises at least text recognition, in order to determine the at least one item of patient information.

This has the advantage that it is possible to capture data in a digitized manner directly from a screen of a medical device. Data can therefore also be captured from those devices which themselves do not have a digital interface for providing the data. The capture may accordingly comprise the optical recording and provision of an image of the screen of the external device, in particular also a video recording and/or recording of repeated photographs of the screen. Alternatively or additionally, acoustic signals from the external device, for example signal sounds and/or alarm sounds, can be recorded by the acoustic capture. During processing, a pitch (that is to say frequency) and/or a temporal repetition rate of the acoustic signals can then be determined, for example, in order to determine the category and/or a value of the patient information. The categories are, for example, an alarm and/or a vital sign (such as pulse) or the like. The value is the specific value of the pulse, for example. In the same manner, the category and/or the value of the patient information can also be determined by the text recognition on the basis of the text recognized in the process.

As a further step, provision may be made to convert the determined patient information into a medical data format in order to provide the patient information, preferably in the device according to the invention and/or for a further functional component and/or in at least one information system, preferably in a digital form. The conversion can be into a Fast Healthcare Interoperability Resources (FHIR) data format, for example. It may be possible for the converted patient information to be transmitted to at least one information system such as an external software system via an interface. The conversion can be carried out, for example, by the device according to the invention and/or by a data processing system.

Provision may be made for the capture to be carried out in the form of an optical capture of a screen of the external device. The connection of medical devices to the hospital IT is conventionally complex and time-consuming. Older models of such devices also often do not have a network interface or the like in order to digitally provide the data. In practice, the vital data such as pulse or oxygen saturation are usually still manually noted from the screen of the external device for this reason. In contrast, the optical and/or acoustic determination makes it possible to determine the data as patient information directly from the screen of the external device and/or acoustically. In this case, the external device may not have any mechanical and/or data connection to the device according to the invention.

Provision may be made for the optical capture to be carried out by an optical interface and/or for the acoustic capture to be carried out by an acoustic interface. The respective interface may be in the form of a stationary and/or electronic interface. Furthermore, the interface may be designed to be stationary by virtue of the fact that the interface is fastened outside the provided device according to the invention for stationary use at the hospital bed of the intensive-care unit. The fastening is carried out, for example, at the intensive-care unit and/or at a hospital bed of the intensive-care unit by means of screws or adhesives. For example, the optical interface is in the form of a stationary camera. The stationary camera may be fastened outside the device provided. The image information obtained may be provided for processing by means of radio transmission. It is conceivable for the radio transmission to be carried out from the optical and/or acoustic interface to the provided device or the device according to the invention. Alternatively or additionally, the radio transmission may also be carried out from the optical and/or acoustic interface to an external data processing system such as a server. The radio transmission is carried out, for example, in the form of Bluetooth or WLAN (Wireless Local Area Network) or NFC (Near Field Communication) communication or the like. The processing may optionally be carried out by the data processing system, and the patient information provided thereby may be directly transmitted from the data processing system to an information system such as a database of the hospital.

It may also be possible for the interface or the camera to be fastened on a ceiling of the intensive-care unit and/or to be aligned with the external device. During processing, the image information can be transformed on the basis of the alignment in order to prepare for the text recognition. It is therefore possible to carry out perspective equalization and to therefore compensate for the alignment of the camera.

It is also possible for the processing to comprise the following steps which are carried out, in particular, by a computer program and/or by an algorithm such as an artificial neural network or another machine-trained algorithm:
detecting a screen of the external device in the image information, for example by means of edge detection or pattern recognition or by using artificial intelligence such as a neural network or by detecting the contours of the screen,
trimming the image information on the basis of the detected screen,
detecting edges and/or transforming the detected screen in order to preferably carry out perspective equalization of the image information, with the result that the text recognition is carried out on the basis of the perspectively equalized image information.

In addition, the processing may comprise the following steps:
carrying out the text recognition (in particular an “Optical Character Recognition”, OCR for short) on the basis of the image information, in particular on the basis of the equalized and/or trimmed image information,
assigning the recognized text to a category of the patient information, wherein the category can preferably be selected from one of the following categories: one or more vital data type(s) such as a blood pressure or a pulse or an oxygen saturation or a blood glucose value, a measured value from a ventilator, an alarm.

Provision may be made for the patient information to comprise at least one vital parameters and/or at least one item of output information, in particular an item of information output on a screen, of the external medical device and/or at least one alarm of the external medical device and/or at least one measured value determined and output by the external medical device, wherein the external device may be in the form of a vital data monitor or a ventilator. The connection of such external devices to the device according to the invention may significantly assist in the treatment.

Text recognized by the text recognition may be advantageously assigned to a category of the patient information, in particular by evaluating an item of color information in the image information and/or a device type of the external medical device on the basis of the image information. For example, the device type of the external device can be determined during processing on the basis of the image information by means of pattern recognition and/or by using artificial intelligence such as a neural network or the like. The evaluation of the color information may be designed to recognize the color of the text. In this case, use is made of the fact that different vital data are often represented in different colors.

The text displayed on the external device may also be represented in one or more, in particular predefined, colors. This color of the text can then be determined during processing on the basis of the color information in the image information in order to assign the text to the category. This category of the text then also corresponds to the category of the patient information. The patient information provided can then be indicated with this category.

It is also conceivable for the recognized text to be assigned to a category in at least two stages. According to a first stage, the category can be preselected by identifying the device type of the external device on the basis of the image information. For example, when the device type “vital data monitor” is identified, the available categories for the preselection are restricted to vital parameters (comprising, for example, blood pressure and/or pulse and/or oxygen saturation and/or the like). According to a second stage, the category of the text can then be determined from the preselected categories by evaluating a text structure and/or semantic text content (that is to say, for example, the numerical value) and/or an item of color information and/or a position of the text from the image information or the text. For example, the category can be determined as a specific vital parameter, blood pressure or pulse, on the basis of the identified color of the text and/or the position of the text.

A closure cap for covering a camera, in particular a camera lens, is also disclosed. The device according to the invention may have a housing on which the closure cap can be arranged. The closure cap preferably has an elongated body. A connection element is arranged outside a center of the body. The connection element is designed to (attractively) magnetically interact with a cooperative connection element arranged beside the camera or the camera lens, for example on the housing of the device according to the invention. The connection element comprises, for example, a magnet or a ferromagnetic material. The cooperative connection element is configured such that the connection element can magnetically connect to the cooperative connection element. That is to say, the cooperative connection element comprises a magnet if the connection element has the ferromagnetic material. If the connection element has the magnet, the cooperative connection element comprises a ferromagnetic material or a further magnet which is arranged in such a manner that the opposite poles of the magnet and of the further magnet are opposite one another if the closure cap covers the camera or the camera lens or is arranged on the housing.

As a result of the fact that the connection element is arranged outside the center of the closure cap, the closure cap can be pivoted in front of the camera or the camera lens (closed state) by rotating about the connection element in order to cover the camera or the camera lens or can be pivoted out of the field of view of the camera (open state) in order to uncover the camera or the camera lens. In this case, the magnetic interaction between the connection element and the cooperative connection element permanently remains. Alternatively, the magnetic interaction between the connection element and the cooperative connection element can be released and can be restored in the correct position of the closure cap. That is to say, the closure cap is released from the housing in the open state, for example, and is fitted to the housing again in the closed state.

The idea of the closure cap is to comply with the desire for privacy of patients and medical personnel. Since the advent of integrated cameras in laptops, it is a common phenomenon that the cameras are sealed up using any adhesive slips of paper or the like. This phenomenon does not spare cameras in medical devices either. However, the adhesives often leave residues on the camera or the camera lens, thus impairing the image quality. However, in medical devices, this may have consequences for the health of the patient if the camera can no longer correctly record an external device, for example. In this respect, the closure cap makes it possible to preserve the privacy of patients and medical personnel if the camera is not required and ensures unrestricted functionality of the camera if it is required.

In this respect, the closure cap can be advantageously used with the device according to the invention if the device according to the invention has a camera. The cooperative connection element can then be arranged beside the camera (lens) in the housing of the device according to the invention. The closure cap may also be part of a system according to the invention.

### EXEMPLARY EMBODIMENTS

The invention is explained in more detail on the basis of exemplary embodiments in the drawing, in which in each case in a schematic illustration:

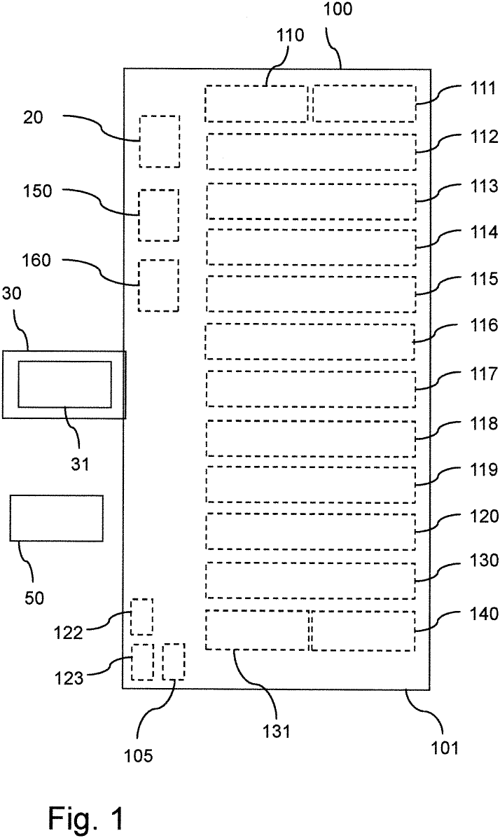

FIG. 1 shows a device according to the invention and a system according to the invention,

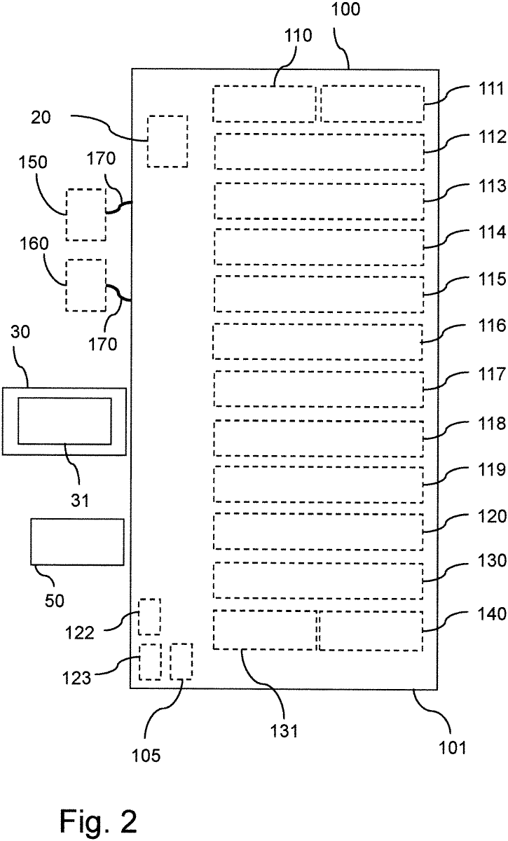

FIG. 2 shows a device according to the invention and a system according to the invention in accordance with a further variant,

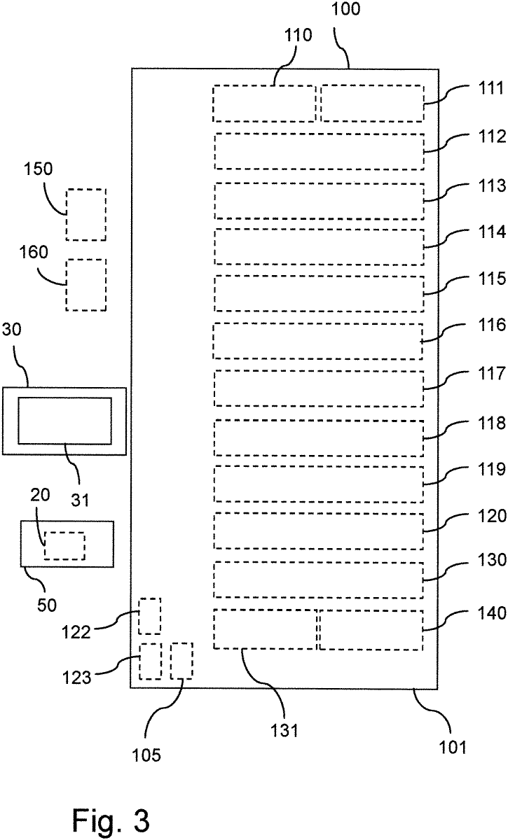

FIG. 3 shows a device according to the invention and a system according to the invention in accordance with a further variant,

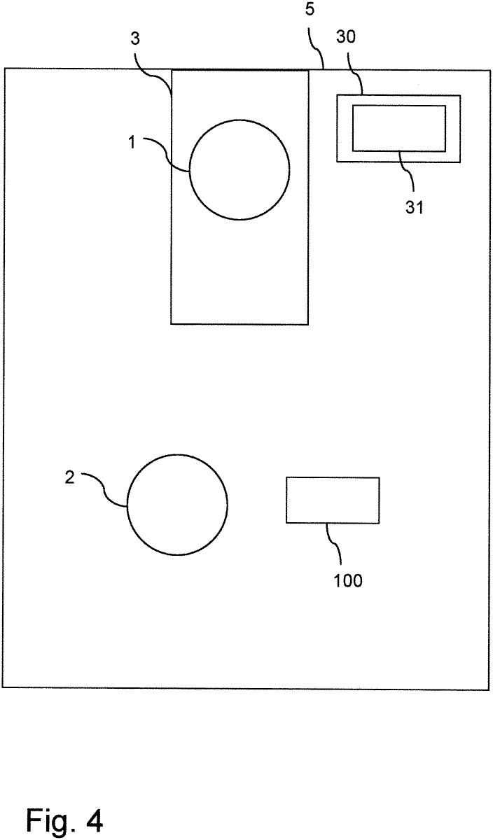

FIG. 4 shows a plan view of a device according to the invention and of a system according to the invention,

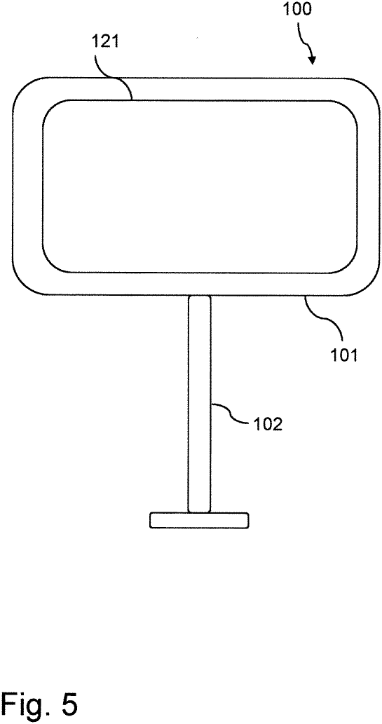

FIG. 5 shows a front view of a device according to the invention and parts of a system according to the invention,

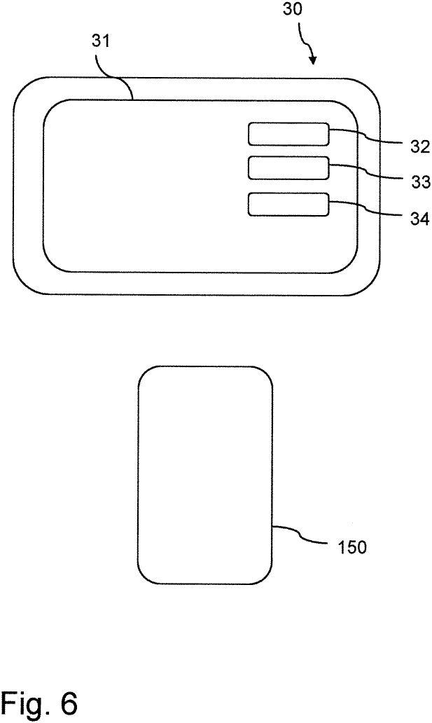

FIG. 6 shows a front view of a device according to the invention and parts of a system according to the invention,

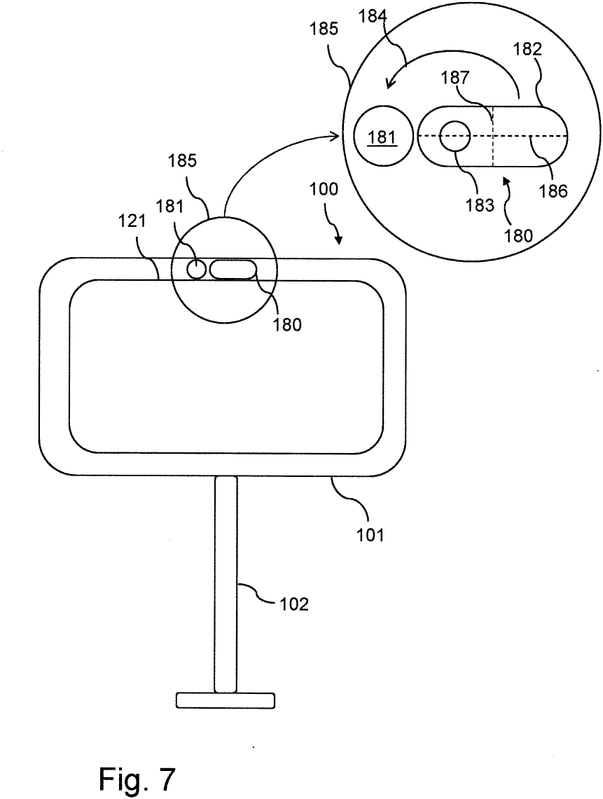

FIG. 7 shows the device according to the invention and parts of the system according to the invention from FIG. 5 in an exemplary embodiment with a closure cap for a camera.

In FIGS. 1 to 3, the device 100 according to the invention comprises an input component 120, a dialog component 130 and at least two of the functional components 111-119 illustrated. If the device 100 is modular, it may also variably have only individual ones of the functional components 111-119. In order to visualize the variable structure of the device 100, the functional components 111-119 are each illustrated using dashed lines. The functional components 111-119 may each have electronic components which have, for example, at least one processor for executing instructions of a computer program. Electronic interfaces of the functional components 111-119, which allow data to be transmitted between the functional components 111-119 and/or with the dialog component 130 and/or the input component 120, may also be provided. This is advantageous, in particular, for enabling the modular configuration of the device 100. The interfaces comprise, for example, conductor tracks and/or cables for transmitting signals. The functional components 111-119 may also each be designed with printed circuit boards on which the electronic components are arranged. In the case of a modular design of the device 100, slots and/or other electronic coupling means (such as releasable plugs or other connection means) may be provided for the printed circuit boards and/or interfaces.

The device 100 according to the invention is designed for assistance in the treatment, in particular medical or intensive-care treatment, of a patient 1. In this case, the device 100 may be specifically designed for this intended use, that is to say the assistance in the (medical) treatment, and may be arranged for this purpose in an intensive-care unit 5, for example in the manner illustrated in FIG. 4. The device 100 may also be adapted for stationary use at the hospital bed 3 of the intensive-care unit 5. This possibly also entails design adaptations of the device 100, for example a disinfectable design of the housing 101 of the device 100 and/or a failsafe redundant design of parts of the functional components 111-119. The design of the device 100 is illustrated in more detail in FIG. 5 in which the device 100 according to the invention is designed in a stationary variant with a housing 101 and a stand means 102.

The various functional components 111-119 illustrated in FIGS. 1 to 3 are used to provide different functions for inputting and/or outputting information relating to the treatment. Comprehensive assistance in the treatment is therefore possible. In order to provide some of the information, it is possible to optionally provide an interface 140 which is in the form of part of the device 100 for the purpose of establishing a data connection to external devices 30 and/or at least one information system 50. For the purpose of interchanging data, the interface 140 may be designed using FHIR. In this manner, at least some of the information can be transmitted to the device 100 via the data connection or can be transmitted from the device 100 and stored in the information system 50, for example.

The input component 120 may be designed to capture at least one input by a user 2. For this purpose, the input component 120 has, for example, a touchscreen 121 (see figure and 5) and/or a microphone 122 in order to capture a key and/or text and/or voice input by the user 2 as the input. This input may be forwarded - for example after voice recognition - to the dialog component 130 for dialog-based interaction with the user 2. This makes it possible for the user 2 to operate at least one of the provided functions of the functional components 111-119. The forwarding is carried out, for example, by transmitting data, for example via electronic interfaces and/or an electronic bus system of the device 100. For acoustic output, the input component 120 may have at least one loudspeaker 123 or, for visual output, may have the touchscreen 121 as a screen 121.

It is particularly advantageous if at least one of the functions of the functional components 111-119 is designed to filter and output laboratory values relating to the patient 1. Specifically, the first functional component 111 may be provided for this purpose. A second functional component 112 may be used to provide a second function for documenting the treatment. A third functional component 113 is possibly provided for the purpose of providing a third function for retrieving information from a medical knowledge database 20. For this purpose, the knowledge database 20 may be provided outside the device 100 according to the invention, for example in an information system 50, as shown in FIG. 3. In FIGS. 1 and 2, the knowledge database 20 is alternatively or additionally part of the device 100 according to the invention, that is to say is stored in a non-volatile data memory 105 of the device 100, for example.

It is also possible for a fourth functional component 114 to be provided for the purpose of predicting a medical condition of the patient 1. This “prediction function” can be used to predict a critical (that is to say life-threatening) condition of the patient 1 and can possibly also output a proposed treatment on the basis of an analysis of patient information and/or on the basis of the prediction.

A fifth functional component 115 is also optionally provided in order to assist and/or guide a medical professional and/or to provide documentation during treatment according to a medical guideline.

The fifth functional component 115 and/or at least one further functional component may possibly also be designed to intelligently monitor the documentation function. In this respect, it is possible to provide a reminder, for example, if details are still missing in the documentation. This “control function” may, for example, carry out a comparison with a prestored template which indicates which details must be included in the documentation. This qualitatively ensures that the necessary data are acquired during treatment.

A sixth functional component 116 can be used to use checklists during treatment. A seventh functional component 117 may also be used to at least partially combine the input and/or output information of the previous functions. For this purpose, the information such as the laboratory values and/or the information from the knowledge database 20 and/or the proposed treatment and/or the instructions for assistance and/or guidance and/or the like may be displayed together, for example in a graphical and/or text-based manner, on a display surface of a screen 121.

It is likewise possible for an eighth functional component 118 to be designed to provide a reminder to document the treatment. For example, it may be specified that certain processes must be regularly documented during treatment. The reminder may be output, for example, in the form of an acoustic reminder via the loudspeaker 123 and/or in the form of a visual reminder on the screen 121.

A database may also be provided for the functions and may comprise patient information which is provided, for example, in the data memory 105 of the device 100 and/or in the information system 50. For example, the functional component 118 can check, using the database, whether part of the documentation is missing in order to output a corresponding reminder if the documents are missing. The prediction function may also use, in particular analyze, this database for the purpose of analyzing the patient information and/or predicting the medical condition. The first functional component 111 may determine, on the basis of an analysis of the database, which of the laboratory values should be filtered out and therefore output. The database may therefore provide a reliable decision-making basis for the functions. In this case, data are understood as meaning, in particular, digitally stored and evaluable data.

It is also conceivable for a ninth functional component 119 to be designed to automatically document the treatment on the basis of an analysis of an item of user information that has been input. The analysis can be carried out, for example, on the basis of predefined rules by means of data processing of the user information.

A tenth functional component 110 may also be provided for telemedical assistance and has an interface to at least one network, in particular a communication network.

In addition, an eleventh functional component 131 may be provided for the purpose of optically and/or acoustically determining at least one item of patient information in an environment of the device 100 according to the invention.

It is also possible for an optical interface 150 illustrated in FIGS. 1 to 3 to be provided in order to optically determine at least one item of patient information in an environment of the device 100. Alternatively or additionally, an acoustic interface 160 may be provided in order to acoustically determine at least one item of patient information in an environment of the device 100. The optical and/or acoustic interface 150, 160 may be part of the device 100 according to the invention, according to FIGS. 1 and 2. In this case, the optical and/or acoustic interface 150, 160 is integrated in the housing 101 of the device 100 in FIG. 1, but is provided outside the housing 101 of the device 100 in FIG. 2. In order to also make it possible in the latter case to control the respective interface 150, 160 and/or to transmit the determined patient information, for example, the respective interface 150, 160 may be connected to at least one further component of the device 100 via a cable 170. An alternative is shown in FIG. 3 in which the respective interface 150, 160 is provided completely outside the device 100. The interface 150, 160 is therefore not part of the device 100, but may optionally enable the transmission of data and/or control via a radio interface to the device 100. If the respective optical and/or acoustic interface 150, 160 is independent, it can possibly also be used without using the device 100.

It is possible for the optical interface 150 to have at least one image sensor (not explicitly illustrated) in order to optically capture at least one external medical device 30 in order to determine the patient information on the basis of the optical capture. In this case, the optical interface 150 may have, for example, at least one camera as an image sensor in order to record at least one screen 31 of a monitoring monitor 30 of the intensive-care unit 5 in order to determine the patient information on the basis of an evaluation of the optical recording. The interface 150 may have a data connection to at least one of the functional components 111-119.

The optical interface 150 may also have at least one or precisely one omnidirectional camera or may be in the form of such a camera in order to capture a plurality of external devices 30 at the same time.

FIG. 6 shows that it may be possible to optically determine and provide at least one item of patient information using the eleventh functional component 131 and/or the optical interfaces 150. For this purpose, provision is made, for example, to carry out an optical capture, for example by means of the optical interface 150, during which one or more external medical devices 30 are captured in order to obtain at least one item of image information from the capture. The image information comprises, for example, one or more text fields 32, 33, 34 of a screen 31 of the external device 30. In order to ensure this during treatment of a patient, the optical interface 150 may be fastened in a stationary manner on a ceiling of the intensive-care unit 5 and may be aligned with the screen 31 of the external device 30. The image information obtained can then be processed, wherein the processing comprises at least text recognition in order to determine the at least one item of patient information. The text recognition is carried out, in particular, on the basis of the text fields 32, 33, 34 which may likewise be represented in the image information. For example, a first text field 32 shows a first category of vital data such as a pulse, a second text field 33 possibly shows a second category of vital data such as a blood pressure, and a third text field 34 shows, for example, a third category of vital data such as an oxygen saturation value. In this case, the various text fields 32, 33, 34 may present the text at a different position of the screen and/or in a different color. The processing may comprise the identification of the device type of the external device 30 and/or the color of the text and/or the position in order to determine the category for the text on the basis of this identified information. The text can then be provided as an item of patient information assigned to the category. The determined patient information can optionally also be converted into a medical data format in order to provide the patient information, in particular for an information system 50, that is to say to store it there, for example.

FIG. 7 shows the device 100 according to the invention and parts of the system according to the invention from FIG. 5 in an exemplary embodiment with a camera, the camera lens 181 of which can be covered by means of a closure cap 180. The open state is illustrated in FIG. 7. The section 185 which shows the camera lens 181 and the closure cap 180 is illustrated again as an enlargement in a detailed view in the top right-hand corner.

The closure cap 180 has an elongated body 182. The body 182 is in the form of a rectangle here, the two shorter sides of which are replaced with a semicircle. A body having two central axes 186, 187, in particular central axes perpendicular to one another, is referred to as an elongated body. The elongated body now has a greater extent (length) along the first central axis 186 than along the second central axis 187. The body 182 advantageously has the absence of a ferromagnetic material. The body 182 has a plastic, for example. A connection element 183 is arranged outside a center of the body 182. The center of the body 182 may be characterized, for example, by the point of intersection of the first central axis 186 with the second central axis 187. The connection element 183 is designed to (attractively) magnetically interact with a cooperative connection element (not shown) arranged beside the camera lens 181. The cooperative connection element is behind the connection element and is therefore not visible in the view in FIG. 7.

The connection element 183 now forms an axis of rotation for the closure cap 180. The movement arrow 184 now indicates the possible rotation of the closure cap 180 around the connection element 183. As a result of the rotation, the closure cap 180 can be transferred from the illustrated open state to the closed state (not illustrated).

If the camera is used, the closure cap can therefore be easily opened and, if the camera is not used, the closure cap can be closed again just as easily.

List of reference signs

| | |
|---|---|
| 1 | Patient |
| 2 | User |
| 3 | Hospital bed |
| 5 | Intensive-care unit |
| 20 | Knowledge database |
| 30 | External device |
| 31 | Screen |
| 32 | First text field |
| 33 | Second text field |
| 34 | Third text field |
| 50 | Information system |
| 100 | Device |
| 101 | Housing |
| 102 | Stand means |
| 105 | Data memory |
| 110 | Tenth functional component |
| 111 | First functional component |
| 112 | Second functional component |
| 113 | Third functional component |
| 114 | Fourth functional component |
| 115 | Fifth functional component |
| 116 | Sixth functional component |
| 117 | Seventh functional component |
| 118 | Eighth functional component |
| 119 | Ninth functional component |
| 120 | Input component |
| 121 | Screen |
| 122 | Microphone |
| 130 | Dialog component |
| 131 | Eleventh functional component |
| 140 | Interface |
| 150 | Optical interface |
| 160 | Acoustic interface |
| 170 | Cable |
| 180 | Closure cap |
| 181 | Camera lens |
| 182 | Body of the closure cap |
| 183 | Connection element |
| 184 | Movement arrow |
| 185 | Section |
| 186 | First central axis |
| 187 | Second central axis |

1. A device for assisting in the intensive-care treatment of a patient for stationary use at the hospital bed of an intensive-care unit, comprising:
at least two different functional components for providing different functions for inputting and/or outputting information relating to the treatment,
an input component for at least one input by a user,
a dialog component for dialog-based interaction with the user on the basis of the input in order to operate at least one of the functions provided, wherein
at least one of the functions is designed to filter and output laboratory values relating to the patient.

2. The device as claimed in claim 1, wherein
the following functional components are provided:
a first functional component for providing the function for filtering and outputting the laboratory values relating to the patient as a first function,
a second functional component for providing a second function for documenting the treatment,
a third functional component for providing a third function for retrieving information from a medical knowledge database.

3. The device as claimed in claim 1, wherein
the device is in the form of a stationary, bedside device in order for the user to carry out the input in a manner adjacent to the hospital bed of the patient.

4. The device as claimed in claim 1, wherein
the device has at least one interface to at least one patient data management system, of the intensive-care unit in order to store and/or read out the information at least partially via the information system during input and/or output.

5. The device as claimed in claim 1, wherein
the device has at least one interface to an information system and/or to at least one external sensor in order to provide patient information relating to a condition of the patient.

6. The device as claimed in claim 1, wherein
the input component is designed for the voice recognition of a voice of the user in order to receive the input by voice.

7. The device as claimed in claim 1, wherein
the dialog component is designed to evaluate the input by a neural network.

8. The device as claimed in claim 1, wherein
the function for filtering and outputting laboratory values relating to the patient is designed to carry out the filtering on the basis of patient information in order to automatically filter the laboratory values according to relevance to the treatment.

9. The device as claimed in claim 8, wherein
the function for filtering has a machine-trained evaluation means.

10. The device as claimed in claim 8, wherein
the function for filtering has an evaluation means which has a set of rules for assigning combinations of the patient information to a selection of the laboratory values in order to output the selected laboratory values to the user.

11. The device as claimed in claim 9, wherein
the evaluation means for filtering is designed to detect a future deviation of at least one laboratory value from a normal range in order to output this laboratory value.

12. The device as claimed in claim 1, wherein
one of the functional components is provided for the purpose of providing a function for predicting a medical condition of the patient in order to predict a disease progression of the patient on the basis of patient information.

13. The device as claimed in claim 1, wherein
one of the functional components is provided for the purpose of providing a function for assisting and guiding a medical professional and for documentation during treatment according to at least one medical guideline.

14. (canceled)

15. The device as claimed in claim 1, wherein
at least one of the functional components is designed, in order to provide at least one of the functions, to automatically repeatedly carry out processing for the purpose of determining the information in order to provide the determined information for subsequent retrieval by the input component.

16. The device as claimed in claim 1, wherein
the dialog component is designed to detect a wake-up signal in order to initiate a dialog for operating the functions when the wake-up signal is detected.

17. (canceled)

18. The device as claimed in claim 1, wherein
one of the functional components is designed to automatically document the treatment on the basis of an analysis of an item of user information that has been input.

19. The device as claimed in claim 1, wherein
an optical interface is provided in order to optically determine at least one item of patient information in an environment of the device.

20. (canceled)

21. (canceled)

22. (canceled)

23. The device as claimed in claim 1, wherein
an acoustic interface is provided in order to acoustically determine at least one item of patient information.

24. The device as claimed in claim 1, wherein
one of the functional components is provided for the purpose of providing a proposed treatment on the basis of an analysis of patient information.

25. (canceled)

26. (canceled)

27. (canceled)

28. A method for assisting in the intensive-care treatment of a patient,
wherein the following steps are carried out:
providing a device for stationary use at the hospital bed of an intensive-care unit,
providing different functions for inputting and/or outputting information relating to the treatment by means of the device,
capturing an input by a user by means of the device,
initiating dialog-based interaction with the user by means of the device on the basis of the captured input in order to operate at least one of the functions provided,
wherein at least one of the functions provided filters and outputs laboratory values relating to the patient.

29. (canceled)

30. (canceled)
